(19) United States
(12) Patent Application Publication
(10) Pub. No.: US 2009/0142854 A1
(43) Pub. Date: Jun. 4, 2009

(54) SILANIZING AGENTS COMPRISING A SACCHARIDE END GROUP AND USES THEREOF, IN PARTICULAR FOR THE FUNCTIONALIZATION OF SOLID SUPPORTS

(76) Inventors: Robert Veronique, Domene (FR); Vinet Francoise, Grenoble (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/719,473

(22) PCT Filed: Nov. 15, 2005
(86) PCT No.: PCT/FR2005/002822

§ 371 Date: Aug. 21, 2007

(30) Foreign Application Priority Data
Nov. 16, 2004 (FR) 0412119

(51) Int. Cl. G01N 33/566; C07H 3/06

(52) U.S. Cl. 436/501; 536/123.1; 536/4.1

(57) ABSTRACT

The invention relates to silanizing agents comprising a saccharide end group and to the use thereof for the functionalization of solid supports. The invention also relates to solid supports that have been functionalized by said silanizing agents (glycochips) and to the use of same, such for biological analysis and, in particular, for screening saccharide molecules or proteinaceous ligands of interest.

The present invention relates to silanizing agents comprising a saccharide end group and to their use for the functionalizing of solid supports. The present invention also relates to the solid supports functionalized by these silanizing agents (glycochips) and to their use, in particular for biological analysis and especially for the screening of saccharide molecules or of protein ligands of interest.

The development of DNA chip technologies has made possible a significant advance in programs related to functional genomics. This is because the miniaturization of techniques for the deposition or synthesis of DNA has resulted in DNA analyses being carried out in parallel, and thus according to multiple parameters, on chips. More recently, the emergence of proteomics has given rise to the concept of protein chips (Zhu and Sydner, Current Op. in Chem. Biol., 2003, 7, 55-63). The latter make possible the analysis in parallel of interactions of protein/ligand type.

More recently still, biological research has taken an interest in “glycomics”, that is to say in the systematic study of carbohydrate/protein interactions. This is because glycoconjugates (that is to say, any molecule having a domain of glycan type, such as glycoproteins, glycolipids, proteoglycans, and more generally any molecule comprising carbohydrates) have a particularly broad functional repertoire. Chemically, these carbohydrates are molecules constructed by the assembling of simple monomeric blocks. These assemblages can be of natural origin, and optionally fractionated, or of synthetic origin. The various functions of the molecules belonging to the family of the carbohydrates is based on the ability of the carbohydrate structures to interact with a very large number of molecules. The analysis of the mechanisms of recognition between carbohydrates and other molecules is a rapidly developing field of research. It should in particular make it possible to result in the design of novel therapeutic molecules and in a better appreciation of the toxicological risks of certain molecules. Currently, there exist few systematic methods which make it possible to produce saccharide molecules. For this reason, the determination of the structural characteristics involved in an interaction between a molecule and a carbohydrate and the characterization of the interaction itself imply the undertaking of lengthy and tedious studies.

It is therefore necessary, to make progress in the knowledge of the mechanisms of interaction between the molecules of saccharide type and their ligands, to be able to screen libraries of molecules of saccharide type with regard to a specific ligand, for example.

This is why it is found today that a novel type of biochip is emerging: various types of glycochip or carbohydrate array or alternatively oligosaccharide array, which constitute a development of the DNA or protein chip concerned with above, have thus been provided by various authors.

These glycochips are either the result of a deposition on a given substrate of a natural or synthetic saccharide substance (ex situ synthesis) or the result of a supported multiparallel synthesis (combinatorial chemistry) of various oligosaccharide sequences (in situ synthesis) representative of the molecular diversity of certain large families of endogenous glucoconjugates, such as heparans, for example.

The invention which will be described below is part of this technology, being particularly well suited to the manufacture of glycochips, by making possible in particular the attachment of saccharide molecules to solid supports according to a preparation process which is simpler to carry out than the known processes of the prior art.

This is because, in the majority of biochips, a spacer arm forms the connection between the surface of the solid support and the end functional unit characterizing the biochip: oligopeptides, oligonucleotides (Osborn H. M. I. et al., Tetrahedron, 1999, 55, 1807-1850; Stetsenko D. A. et al., Bioconjugate Chemistry, 2001, 12, 576-586) or oligosaccharides (U.S. Pat. No. 6,579,725). This spacer can play several roles simultaneously:
it is a bonding molecule, that is to say that it makes it possible to connect the surface of the solid support to a functional molecule (probe);
it is a spatial distancing arm, that is to say that it makes it possible to move the probe molecule away from the surface of the solid support. This is because the proximity of the solid support to the sites for recognition of the target molecules by the probe molecules can impede or prevent probe/target recognition from taking place and can thus be harmful to the sensitivity and to the analytical quality of the biochips. This is particularly true when the probe molecules are small in size, in particular in the case of glycochips.

Numerous spacer arms have been provided to date but these are not entirely satisfactory from a practical viewpoint as they exhibit a number of unresolved disadvantages:
their structure often involves a severe limitation with regard to the choice of the chemical processes which allow them to be attached to the surface of the solid support,
they do not all make it possible to attach probe molecules of saccharide nature,
they are generally so stable after attaching to the surface of the solid support that the cleavage thereof, in order to recover the biological molecule, cannot be readily carried out and can result in damage to the latter or to the support,
many stages are necessary for their chemical synthesis, for their attachment to the surface of the solid support and for their functionalization, which steps are sometimes difficult to carry out and consequently often expensive.

U.S. Pat. No. 6,579,725 describes in particular a spacer arm capable of attaching probe molecules of oligosaccharide nature. However, this spacer arm, although more effective than those present in an even older prior art, does not make it possible to solve at the same time all the disadvantages mentioned above. Mention may in particular be made that its length, its functionality, its reactivity and its hindrance cannot always be generated at will.

The inventors thus aimed to provide novel silanizing agents which make it possible to overcome all the disadvantages cited above. They aimed in particular to provide novel silanizing agents which make it possible to functionalize, in a single step, the surface of a solid support by molecules of saccharide nature, this being achieved according to a process which is simple, reliable and flexible with regard to the nature and the length of the spacer arm and, finally, less expensive than the processes of the prior art.

It is at this juncture that the inventors have developed that which forms the subject matter of the present invention.

A first subject matter of the present invention is thus a silanizing agent comprising a saccharide end functional group, characterized in that it corresponds to the following formula (I):

A-X—B (I)

in which:
A represents a probe molecule of saccharide nature;
X represents a spacer arm composed of a carbon or heterocarbon chain comprising two ends, one of its two ends covalently connecting said spacer arm X to A and the other end covalently connecting said spacer arm X to B, said chain comprising at least one ethylenic unsaturation situated between its two ends, it being understood that said chain cannot comprise several acetylenic unsaturations;
B is a silanized group.

The present invention thus provides a silanized saccharide molecule which can act as an adjustable spacer arm, the various structures of which influence the reactivity of the arm, that is to say its chemical, electrochemical and/or steric behavior.

According to the invention, the probe molecule of saccharide nature constituting the union A of the compounds of formula (I) above can be of natural or synthetic origin and can optionally be protected by one or more protective groups. This probe molecule can in particular be chosen from all saccharide molecules which have to be attached to a support, for example for analytical or diagnostic reasons. It can in particular be synthesized for the purpose of representing a saccharide molecule or biomolecule of biological advantage, such as a heparan sulfate, for example, or for the purpose of representing a saccharide chain itself acting as spacer between a surface and a molecule or biomolecule of biological advantage.

According to an advantageous embodiment of the invention, the probe molecule of saccharide nature exhibits a molecular weight of between 180 and 10 000 g/mol approximately and more preferably still between 360 and 900 g/mol approximately.

It is preferably chosen from:
i) monosaccharides and in particular from glucosamine, azidoglucosamine, D-ribose, D-xylose, L-arabinose, D-glucose, D-galactose, D-mannose, 2-deoxyribose, L-fusose, N-acetyl-D-glucosamine, N-acetyl-D-galactosamine, N-acetylneuraminic acid, D-glucuronic acid, L-iduronic acid, D-sorbitol, D-mannitol, and the like,
ii) oligosaccharides and in particular from sucrose, lactose, fragments of heparan sulfates, saccharide fragments of heparin, of chondroitin and of dermatan sulfates, Lewis antigens, and the like,
iii) polyoligosaccharides and in particular from saccharide fractions of heparan sulfates, of heparin and of chondroitin, dermatan sulfates, and the like,
iv) glycoconjugates and in particular from heparan sulfates, heparin, chondroitin, dermatan sulfates, and the like,
v) glycoproteins, such as immunoglobulin G, hyaluronic acid, and the like,
vi) glycolipids, such as galactosylceramides, gangliosides and cerebrosides, and the like,
viii) glycol lipoproteins, such as the glycoprotein G90 extracted from tissues from the earthworm Eisenia foetida (family of the Lumbricidae) or else the glycoprotein MPB83 supplied by the laboratory “Veterinary Laboratories Agency” (VLA, Weybridge, UK).

One or more of the hydroxyl and/or amine functional groups of the saccharide entities of the probe molecule can be protected by one or more protective groups. These protective groups are well known to a person skilled in the art and are fully described in the work by T. W. Greene et al., “Protective Groups in Organic Chemistry”, Second Edition, A Wiley-Interscience Publication, 1991.

According to an advantageous form of the present invention, these protective groups are chosen from the following groups: acetyl; benzyl; aryl and in particular the aryl groups substituted by an R radical chosen from alkyl chains having from 1 to 40 carbon atoms; 2,2,2-trichloroethyloxycarbonyl (Troc); benzyloxycarbonyl (Z); trichloroacetamidate (TCA); tert-butyloxycarbonyl (BOC) and fluoranylmethoxycarbonyl (Fmoc).

According to another advantageous embodiment of the invention, one or more of the hydroxyl and/or amine functional groups of the saccharide entities of the probe molecule can be substituted by one or more hydrophobic groups which make it possible to render the spacer arm more specific and/or more selective with regard to the target molecule which will become attached to the probe molecule and/or to its role during the use of the spacer arm.

Mention may in particular be made of the case where, by protecting the hydroxyl functional groups by protective groups, such as benzyl, acetate, benzylidene, isopropylidene or phthalimide groups and the like, the saccharide part can be rendered more or less hydrophobic.

According to yet another advantageous embodiment of the invention, the anomeric part of the saccharide entities can be functionalized, like any glycoside donor, by a group which will preferably be chosen according to the nature of the covalent bond via which the probe molecule of saccharide nature is attached to one of the two ends of the spacer arm X.

According to an advantageous embodiment of the invention, the covalent bonds via which each of the ends of the chain constituting the spacer arm X are attached to the units A and B result from the reaction between a chemical functional group initially carried by the precursor of the spacer arm X and a complementary chemical functional group carried, on the one hand, by the probe molecule A and, on the other hand, by the silanized group B. There exists, of course, a large number of possible donor/acceptor pairs well known to a person skilled in the art in the field of the chemistry of sugars (Khan S. H. et al., “Modern Methods in Carbohydrate Synthesis”, Perkin Elmer, Applied Biosystems Division, Foster City, Calif., USA, 1996) which can be used to form said covalent bond. Mention may in particular be made, by way of examples, of the covalent bonds resulting from the reaction between a hydroxyl radical and a group chosen from halogen atoms, such as chlorine, bromine, iodine or fluorine, and from phosphite, trichloroacetamidate, thioalkyl, phosphate, pentenyl, sulfoxide and xanthate groups.

The spacer arm X of the compounds of formula (I) in accordance with the invention can be variable in length and in structure. As was seen above, however, it nevertheless always comprises at least one ethylenic unsaturation on the chain directly connecting A and B.

According to a particularly preferred embodiment of the invention, the spacer arm X represents a linear or branched C₂-C₄₀ alkyl or C₆-C₄₀ aryl chain, said chain comprising at least one ethylenic unsaturation and optionally being able to be interrupted by one or more heteroatoms chosen from oxygen, nitrogen, sulfur and silicon and/or one or more functional groups, such as amide, oxime and tertiary amine functional groups, and/or optionally substituted by one or more substituents (preferably from 1 to 10 substituents) chosen from linear or branched C₂-C₂₀ alkyl or C₆-C₂₀ aryl chains, it being possible for said chains optionally also to be interrupted by one or more heteroatoms chosen from oxygen, nitrogen, sulfur and silicon.

According to the invention, the silanized group B is preferably chosen from —Si(R₁)₃, —SiR₁(R₂)₂ and —SiR₁R₂R₃ groups in which the R₁, R₂ and R₃ radicals represent, independently of one another, a halogen atom, such as fluorine or chlorine, a C₁-C₄ alkoxy radical, a C₁-C₄ alkyl radical, an amino radical or an ester functional group.

Preference is very particularly given, among the alkoxy radicals defined for the R₁, R₂ and R₃ radicals, to the methoxy and ethoxy radicals and preference is very particularly given, among the alkyl radicals defined for the R₁, R₂ and R₃ radicals, to the methyl and ethyl radicals.

Mention may in particular be made, among the ester functional groups defined for the R₁, R₂ and R₃ radicals, of the acetoxy radical.

Mention may in particular be made, as silanized end unit B, of the trimethoxysilyl, triethoxysilyl, trimethylsilyl and triethylsilyl groups.

Preference is particularly given, among the silanizing agents of formula (I) above, to those in which:
A is chosen from monosaccharides, oligosaccharides and polysaccharides and more particularly still from oligomers such as Glc-Glc-Glc, Lac-Lac or Gal-Gal-Gal-Gal-Gal in which the abbreviations Glc, Lac and Gal respectively indicate glucose, lactose and galactose;
X represents a carbon chain having from 2 to 40 carbon atoms comprising at least one ethylenic unsaturation, said chain being linear or branched and optionally interrupted by one or more rings and/or one or more functional groups, such as amide, oxime and tertiary amine functional groups;
B represents a trimethoxysilyl or triethoxysilyl group.

Mention may be made, as compound of formula (I) which is very particularly preferred, of the compounds corresponding to the following formulae (I-1) and (I-2):

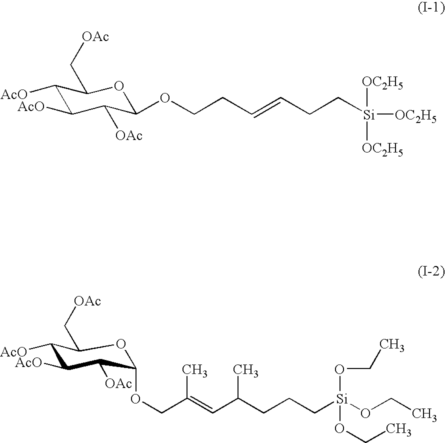

in which Ac represents the acetyl group.

The silanizing agents of formula (I) above can be easily prepared according to the principles of organic synthesis well known to a person skilled in the art according to the nature of the units A, X and B.

In particular, these silanizing agents can generally be prepared by a simple assembling of the units A, X and B, said units being either prepared beforehand or available commercially, it being understood that said units comprise chemical functional groups appropriate for the formation of a covalent bond, on the one hand, between the unit A and one of the ends of the spacer arm X and, on the other hand, between the unit B and the other end of the spacer arm X.

The reactions carried out are generally conventional glycosylation reactions of A with X, followed by hydrosilylation (for example Karstedt) reactions to attach B.

The silanizing agents of formula (I) in accordance with the invention can be used for the functionalization of solid supports.

The subject matter of the present invention is thus the use of at least one silanizing agent of formula (I) as defined above for the functionalization of solid supports and in particular for the manufacture of glycochips.

The use of silanizing agents of formula (I) advantageously makes it possible to rapidly modify the surface of solid supports by a stable layer carrying probe molecules of saccharide nature which can be easily cleaved from the support in view of the presence of at least one ethylenic unsaturation on the spacer arm X of the compounds of formula (I) in accordance with the invention.

When they are used in particular for the preparation of glycochips, the compounds of formula (I) in accordance with the invention exhibit the following advantages:
They act first of all as a spacer arm, making it possible to move the saccharide chain away from the surface of the solid support which supports this chain. The unit X of the compounds of formula (I) in accordance with the invention makes it possible in particular to bond to a very broad range of saccharides, oligosaccharides or polysaccharides. In particular, the compounds of formula (I) can be used either as spacer arm for a first saccharide unit, for example an oligosaccharide unit, which will be attached to the unit X and which it is subsequently possible to increase in size (combinatorial chemistry on a solid support), or for the attaching of presynthesized saccharide probe molecules (attaching to the unit X, at the anomeric position of their reducing part).
The spacer arm can be cleaved: by virtue of the presence of at least one ethylenic unsaturation on the unit X of the compounds of formula (I) which it is possible to readily open in targeted fashion in order to isolate the sugar from the solid phase, this being done under conditions which do not in any way detrimentally affect the integrity of the saccharide probe molecule. Use may in particular be made, as cleavage method, of ozonolysis, Grubbs metathesis or the method involving dihydroxylation by osmylation followed by oxidative cleavage of the diol (OsO₄, NaIO₄), and also other gentle chemical cleavage reactions known to a person skilled in the art.
Finally, because of their chemical functionalities and the choice of the substituents on their saccharide backbone, the compounds of formula (I) in accordance with the invention remain inert under many experimental conditions during the reactions carried out.

Another subject matter of the present invention is a process for the preparation of a solid support functionalized by probe molecules of saccharide nature, characterized in that it comprises at least one stage of silanizing at least one surface of a solid support with a solution of at least one silanizing agent of formula (I) in an organic solvent.

The organic solvent is preferably chosen from trichloroethylene, toluene and lower alcohols, such as ethanol or methanol, these solvents optionally having added to them a basic compound, such as triethylamine or N,N-diisopropylethylamine (DIEA).

The operation in which the solid support is brought into contact with the solution of the silanizing agent of formula (I) is preferably carried out at a temperature of between 4 and 80° C. approximately for 1 to 48 hours approximately.

The substrate is subsequently rinsed with the reaction solvent or with chloroform and then dried, preferably with nitrogen.

This process exhibits the advantage of being simple to carry out and of combining the stage of silanizing with the stage of functionalizing the solid support, whereas the known processes of the prior art required at least three successive stages, that is to say a first stage of silanizing the surface of the solid support with a molecule having a functional group which makes possible the attachment of a spacer arm in a second stage and, finally, the attachment of a saccharide probe molecule in a third stage, for example according to a glycosylation reaction. In this case, an inactivating stage (capping of the nonglycosylated sites) was then necessary, which is not, in contrast, the case according to the process of the present invention.

The solid supports which can be functionalized by the silanizing agents of formula (I) in accordance with the invention are preferably chosen from supports based on glass, on silica or on any other material known to a person skilled in the art as being able to be silanized.

These solid supports have at least one flat or nonflat and smooth or structured surface and can, for example, be provided in the form of a slide, flat plate, plate with wells, capillary or porous or nonporous bead.

Another subject matter of the present invention is thus solid supports, characterized in that they comprise at least one surface functionalized by one or more silanizing agents of formula (I) as defined above.

Such supports constitute glycochips which are, for example, capable of being used for the identification, by screening, of saccharide molecules and in particular of oligosaccharide sequences which recognize a specific protein of advantage, for example using the method described in international application WO-A-03/008927.

Conversely, the glycochips in accordance with the present invention can also be used for the identification, by screening, of ligands, for example of protein ligands which recognize a saccharide of advantage.

Consequently, a final subject matter of the present invention is a process for screening saccharide molecules and in particular oligosaccharide sequences or respectively protein ligands, characterized in that it comprises at least one stage in which a solid support comprising at least one surface functionalized by at least one silanizing agent of formula (I) as defined above is brought into contact with a solution including one or more potential oligosaccharide molecules or respectively one or more potential protein ligands.

In these specific applications, the functionalized solid supports in accordance with the present invention make it possible to optimize the screening processes and thus to have available more effectively and more rapidly molecules with a therapeutic or biotechnological aim.

In addition to the preceding provisions, the invention also comprises other provisions which will emerge from the description which will follow, which refers to an example of the preparation of a compound of formula (I) in accordance with the invention and to an example of the functionalization of a solid support with a compound of formula (I) in accordance with the invention.

It should be clearly understood, however, that these examples are given solely by way of illustration of the subject matter of the invention, of which they do not under any circumstances constitute a limitation.

### EXAMPLE 1

### Preparation of the Compound of Formula (I-1)

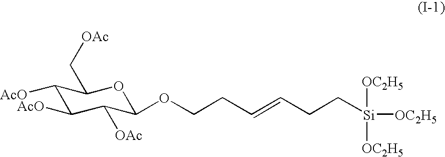

in which Ac represents acetyl.
1) First stage: Preparation of a Spacer Arm (3) and of a Glucose Derivative (5)

The spacer arm (3) is prepared according to the following reaction scheme A:

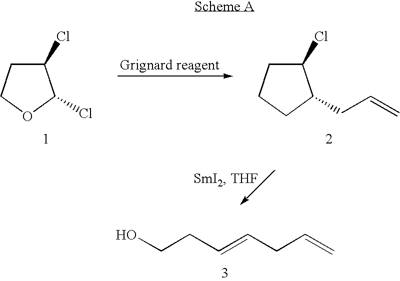

The spacer arm (3) is obtained in 2 successive stages: 2-vinyl-3-chlorotetrahydrofuran (2) is accessible from 2,3-dichlorotetrahydrofuran (1) by treatment with a Grignard reagent according to the process described by L. Crombie and R. D. Wyvill, Journal of the Chemical Society, 1985, Perkin Trans. 1, 1971, and references cited.

The compound (2) is subsequently treated to reflux in tetrahydrofuran for 5 to 165 hours in the presence of 4-7 equivalents of samarium diiodide (SmI₂), according to the process described by L. Crombie and L. J. Rainbow, 1988, Tetrahedron Letters, 29(49), 6517.

The compound (3) is obtained with a yield of 93%.

The glucose derivative (5) is prepared according to the following reaction scheme B:

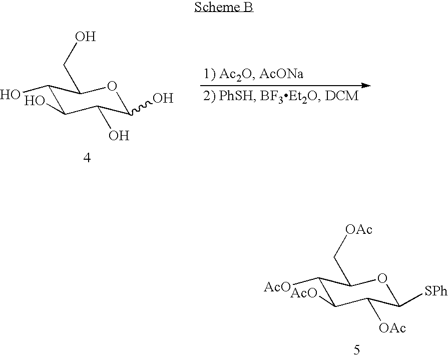

The glucose derivative (5) (thioglycoside) is obtained from D-glucose in two successive stages which are conventional reactions of the chemistry of sugars:
a first stage of peracetylation in an acetic anhydride/sodium acetate (AC₂O/AcONa) medium at 120° C. for 1 hour (the product obtained after precipitation from ice-cold water is recrystallized from 95% ethanol), according to the process described by K. Takeo, Carbohydrate Research, 1980, 87, 147. The peracetylated byproduct is obtained with a yield of 90% and the configuration of the anomeric position is predominantly β;
a second stage of activation of the anomeric position in anhydrous dichloromethane in the presence of thiophenol and of a Lewis acid, in this case boron trifluoride etherate (BF₃.Et₂O), at ambient temperature for one hour, according to the process described by A. K. Choudhury and N. Roy, Synthetic Communications, 1996, 26, 3937. This thioalkylation makes it possible to quantitatively achieve the thioglycoside (5) after recrystallization from methanol. The thiophenyl group is found in the β position due to the presence of a participating group in the 2 position.
   2) Second stage: Preparation of a Glycosylated Spacer Arm (6)

This glycosylated spacer arm is prepared according to the following reaction scheme C:

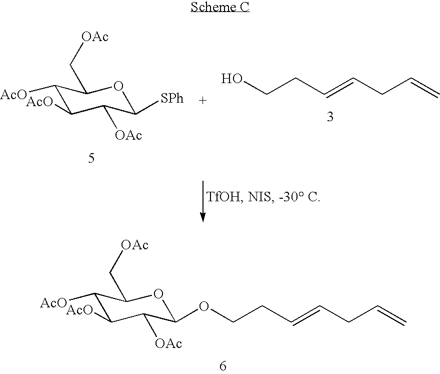

The coupling of these two molecules is a glycosylation reaction of a protected glucose with an unsaturated chain.

The thioglycoside (5) (500 mg, 1.13 mmol, 1 eq.) and the unsaturated spacer arm (3) (127 mg, 1.13 mmol, 1 eq.) are dissolved in anhydrous dichloromethane in the presence of molecular sieve (630 mg). This mixture is kept stirred at ambient temperature for 30 minutes and then the reaction medium is brought to a temperature of −30° C. N-Iodosuccinimide (NIS) (510 mg, 2.26 mmol, 2 eq.) is then added, followed by trifluoromethanesulfonic acid (30 μl, 0.34 mmol, 0.15 eq./NIS). The mixture is then slowly brought back to ambient temperature, with stirring for 30 minutes. The reaction medium is subsequently neutralized with a saturated aqueous sodium hydrogencarbonate solution and then filtered through celite. The organic phase is extracted and washed successively with water, with a saturated aqueous sodium thiosulfate solution and with a saturated aqueous sodium chloride solution. The combined organic phases are dried over magnesium sulfate, filtered and concentrated under vacuum. The solid residue obtained is purified by chromatography on a column of silica gel. The product (6) is obtained with a yield of 70%.

### 3) Third Stage: Preparation of the Compound of Formula (I-1)

This stage is carried out according to the following reaction scheme D:

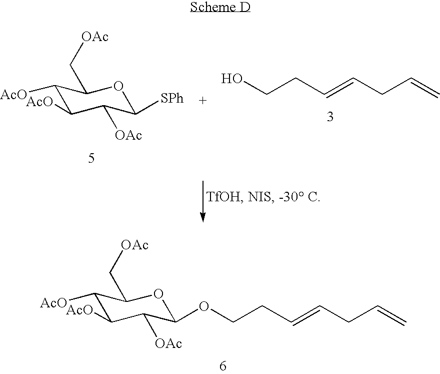

During this stage, 100 mg of the compound (5) obtained above in the preceding stage (2.26×10⁻⁴ mol, 1 eq.) were reacted with 71 μl (3.62×10⁻⁴ mol, 1.6 eq.) of triethoxysilane in the presence of 2.84 μl (4.5×10⁻⁴ mol) of Karstedt catalyst for 3 hours at a temperature of 50° C. approximately in order to result in a colorless liquid of compound of formula (6) with a yield of 60%.

### EXAMPLE 2

### Preparation of a Solid Support Functionalized with a Silanizing Agent of Formula (I)

In this example, a flat substrate made of SiO₂ was used.

### 1) Rehydration of the Substrate (Brown Rehydration)

An SiO₂ substrate was dipped for 2 hours at ambient temperature in a mixture of deionized water (15 ml) and of absolute ethanol (20 ml) including 5 g of NaOH.

The substrate was subsequently washed with deionized water and then it was dipped for 1 hour in 0.2N hydrochloric acid. After dipping, the substrate was again washed with deionized water and then dried in an oven at a temperature of 80° C. for 30 minutes.

### 2) Silanizing of the Substrate by the Compound of Formula (I-1)

The rehydrated substrate was dipped in 10 ml of a trichloroethylene (TCE) solution including 10 mM (21 mg) of compound of formula (I-1) as prepared in example 1 above. After 1 night at ambient temperature, the substrate was silanized by the compound of formula (I-1) in accordance with the invention.

The substrate thus functionalized was subsequently washed with TCE, with ethanol and finally with chloroform. It was subsequently dried in an oven for 30 minutes at a temperature of 50° C.

For the purpose of subsequent use, the functionalized substrate was stored under an inert atmosphere (argon or nitrogen).

This substrate can subsequently be used for the preparation of a glycochip (growth of oligosaccharides on the silanized substrate thus prepared) or of any other molecule or biomolecule chip (in this case, the sugar-comprising silane which functionalizes the substrate becomes a spacer for the attachment of any new molecule or biomolecule, natural or synthetic).

1. A silanizing agent comprising a saccharide end functional group, characterized in that it corresponds to the following formula (I):
A-X—B (I)
in which:
the unit A represents a probe molecule of saccharide nature;
the unit X represents a spacer arm composed of a carbon or heterocarbon chain comprising two ends, one of its two ends covalently connecting said spacer arm X to A and the other end covalently connecting said spacer arm X to B, said chain comprising at least one ethylenic unsaturation situated between its two ends, it being understood that said chain cannot comprise several acetylenic unsaturations;
B is a silanized group.

2. The silanizing agent as claimed in claim 1, characterized in that the probe molecule of saccharide nature exhibits a molecular weight of between 180 and 10 000 g/mol.

3. The silanizing agent as claimed in claim 1, characterized in that the probe molecule of saccharide nature is chosen from monosaccharides, oligosaccharides, polysaccharides, glycoconjugates, glycoproteins, glycolipids and glycolipoproteins.

4. The silanizing agent as claimed in claim 3, characterized in that the monosaccharides are chosen from glucosamine, azidoglucosamine, D-ribose, D-xylose, L-arabinose, D-glucose, D-galactose, D-mannose, 2-deoxyribose, L-fucose, N-acetyl-D-glucosamine, N-acetyl-D-galactosamine, N-acetylneuraminic acid, D-glucuronic acid, L-iduronic acid, D-sorbitol and D-mannitol.

5. The silanizing agent as claimed in claim 3, characterized in that the oligosaccharides are chosen from sucrose, lactose, fragments of heparan sulfates, saccharide fragments of heparin, of chondroitin or of dermatan sulfates, and Lewis antigens.

6. The silanizing agent as claimed in claim 3, characterized in that the polyoligosaccharides are chosen from saccharide fractions of heparan sulfates, of heparin or of chondroitin, and dermatan sulfates.

7. The silanizing agent as claimed in claim 3, characterized in that the glycoconjugates are chosen from heparan sulfates, heparin, chondroitin and dermatan sulfates.

8. The silanizing agent as claimed in claim 3, characterized in that the glycoproteins are chosen from immunoglobulin G and hyaluronic acid.

9. The silanizing agent as claimed in claim 3, characterized in that the glycolipids are chosen from galactosylceramides, gangliosides and cerebrosides.

10. The silanizing agent as claimed in claim 1, characterized in that one or more of the hydroxyl and/or amine functional groups of the saccharide entities of the probe molecule are protected by one or more protective groups chosen from acetyl, benzyl and aryl, 2,2,2-trichloroethyloxycarbonyl, benzyloxycarbonyl, trichloroacetamidate, tert-butyloxycarbonyl and fluoranylmethoxycarbonyl groups.

11. The silanizing agent as claimed in claim 1, characterized in that one or more of the hydroxyl and/or amine functional groups of the saccharide entities of the probe molecule are substituted by one or more hydrophobic groups chosen from benzyl, acetate, benzylidene, isopropylidene and phthalimide groups.

12. The silanizing agent as claimed in claim 1, characterized in that the covalent bonds via which each of the ends of the chain constituting the spacer arm X are attached to the units A and B result from the reaction between a chemical functional group initially carried by the precursor of the spacer arm X and a complementary chemical functional group carried, on the one hand, by the probe molecule A and, on the other hand, by the silanized group B.

13. The silanizing agent as claimed in claim 12, characterized in that said covalent bonds result from the reaction between a hydroxyl radical and a group chosen from halogen atoms and phosphite, trichloroacetamidate, thioalkyl, phosphate, pentenyl, sulfoxide and xanthate groups.

14. The silanizing agent as claimed in claim 1, characterized in that the spacer arm X represents a linear or branched C₂-C₄₀ alkyl or C₆-C₄₀ aryl chain, said chain comprising at least one ethylenic unsaturation and optionally being able to be interrupted by one or more heteroatoms chosen from oxygen, nitrogen, sulfur and silicon and/or one or more functional groups chosen from amide, oxime and tertiary amine functional groups and/or optionally substituted by one or more substituents chosen from linear or branched C₂-C₂₀ alkyl or C₆-C₂₀ aryl chains, it being possible for said chains optionally also to be interrupted by one or more heteroatoms chosen from oxygen, nitrogen, sulfur and silicon.

15. The silanizing agent as claimed in claim 1, characterized in that the silanized group B is chosen from —Si(R₁)₃, —Si(R₁(R₂)₂ and —SiR₁R₂R₃ groups in which the R₁, R₂ and R₃ radicals represent, independently of one another, a halogen atom, a C₁-C₄ alkoxy radical, a C₁-C₄ alkyl radical, an amino radical or an ester functional group.

16. The silanizing agent as claimed in claim 15, characterized in that the silanized group is chosen from the trimethoxysilyl, triethoxysilyl, trimethylsilyl and triethylsilyl groups.

17. The silanizing agent as claimed in claim 1, characterized in that it is chosen from the compounds of formula (I) in which:
A is chosen from monosaccharides, oligosaccharides and polysaccharides,
X represents a carbon chain having from 2 to 40 carbon atoms comprising at least one ethylenic unsaturation, said chain being linear or branched and optionally interrupted by one or more rings and/or one or more functional groups, such as amide, oxime and tertiary amine functional groups;
B represents a trimethoxysilyl or triethoxysilyl group.

18. The silanizing agent as claimed in claim 1, characterized in that it is chosen from the compounds of the following formulae (I-1) and (I-2): 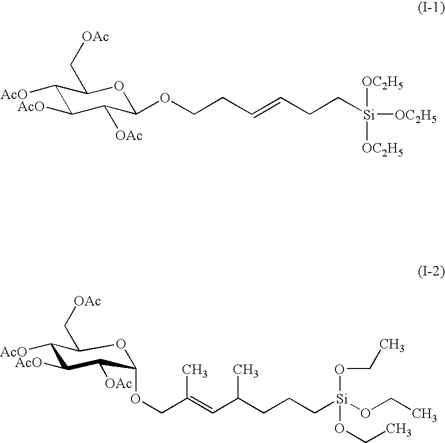
in which Ac represents the acetyl group.

19. The use of at least one silanizing agent of formula (I) as defined in claim 1, for the functionalization of solid supports.

20. The use of at least one silanizing agent of formula (I) as defined in claim 1, for the manufacture of glycochips.

21. A process for the preparation of a solid support functionalized by probe molecules of saccharide nature, characterized in that it comprises at least one stage of silanizing at least one surface of a solid support with a solution of at least one silanizing agent of formula (I) as defined in claim 1, in an organic solvent.

22. A solid support, characterized in that it comprises at least one surface functionalized by one or more silanizing agents of formula (I) as defined in claim 1.

23. The use of a solid support as defined in claim 22 for the identification, by screening, of oligosaccharide sequences which recognize a protein of advantage or of ligands which recognize a saccharide of advantage.

24. A process for screening saccharide molecules or respectively protein ligands, characterized in that it comprises at least one stage in which a solid support as defined in claim 22 is brought into contact with a solution including one or more potential oligosaccharide molecules or respectively one or more potential protein ligands.
